(19) United States
(12) Patent Application Publication
(10) Pub. No.: US 2011/0257610 A1
(43) Pub. Date: Oct. 20, 2011

(54) Therapeutic Diffusion Wound Dressing and Filling Method

(76) Inventor: Amie B. Franklin, Woodbury, MN (US)

(21) Appl. No.: 13/069,810

(22) Filed: Mar. 23, 2011

Related U.S. Application Data
(60) Provisional application No. 61/316,714, filed on Mar. 23, 2010.

(51) Int. Cl. A61M 35/00; B65B 65/00

(52) U.S. Cl. 604/305; 141/4

(57) ABSTRACT

A therapeutic dressing has a permeable bottom layer, a non-permeable upper layer sealed about the periphery to the permeable layer to form at least one reservoir, an absorbent patch integrated with the permeable layer, a hermetic seal attached to the underside of the permeable layer and a filling interface in communication with the reservoir. The dressing is filled with a therapeutic agent and wherein such agent is subsequently diffused through the permeable layer onto the skin of a patient.

### CROSS-REFERENCE TO RELATED APPLICATIONS

This application is a non-provisional of provisional application U.S. 61/316,741, filed Mar. 23, 2010, disclosure is herein incorporated by reference in its entirety.

### BACKGROUND OF THE INVENTION

1. Field of the Invention

The present invention is in the field of medical technologies and pertains more particularly to treatment of tissue via a diffusion type device or dressings, the invention pertaining to unique construction of such dressings and maintenance thereof.

2. Discussion of the State of the Art

In the medical field it is often required to treat tissue of patients in a manner which requires an extended period of bandaging or covering with medication-bearing patches or like devices that work to keep tissue moist, promote healing, to prevent maceration and possible infection, or to prevent any jolting or other physical disruption of a particular tissue area.

One example of such a therapeutic device is a diffusion dressing or device typically applied over a wound or graft to promote new skin growth in an area where the original skin was lost or removed by surgery. Such devices have different names in the art but may be technically referred to as therapeutic diffusion devices because of an active delivery (diffusion) of some therapeutic agent, often delivered in the form of a gas through an interfacing diffusion layer of the dressing or device to the affected tissue over time.

A diffusion device for treating tissue is typically charged with some therapeutic agent that is expelled from the device through a diffusion process one effect there being that the dressing eventually becomes depleted of agent and is no longer therapeutic to the tissue being treated. Most such diffusion devices employ gasses infused into a reservoir in the device, the gases being diffused into a tissue area through a special diffusion layer of the device. Diffusion layers and adhesive layers for application of the dressing over a wound are typically a part of such apparatus.

It would be desirable to manage a diffusion dressing with therapeutic agent both at the pre-application level and during active treatment of a wound more efficiently and with less waste of therapeutic agents.

Therefore, what is clearly needed is better apparatus and methods for managing therapeutic agents during diffusion aided healing.

### SUMMARY OF THE INVENTION

A problem stated above is that efficient management of wound dressings including diffusion dressings charged with therapeutic agent is desirable in wound care, but many of the conventional means for charging diffusion dressings with therapeutics such as through a port, also create inefficiency via leakage of therapeutic agents from the wound dressing or from the wound the agents are dispensed to heal. The inventors therefore considered functional elements of a diffusion therapeutic agent delivery system, looking for those that exhibit interoperability that could potentially be harnessed to provide efficient management of therapeutic agents in a diffusion dressing but in a manner that would not create additional work or waste.

Every therapeutic agent delivery system utilizing diffusion dressings is characterized by release of such therapeutic agents onto a wound or wounds of a patient, one by-product of which is an abundance of healing activity in a shorter period of time. Most such therapeutic agent delivery systems employ therapeutic agents under pressure and diffusion type dressings to conduct the therapeutic agents from the containers or canisters to a more realistic point to release the agents onto the wound tissues and pressurized vessels, delivery tubes, and diffusion dressings are typically a part of such apparatus.

The present inventor realized in an inventive moment that if, at the point of application, therapeutic agents could be caused to diffuse through a diffusion dressing in a more efficient and controlled manner, significant improvement of healing and shortening of healing time might result. The inventor therefore constructed a unique therapeutic agent delivery system for wounds that allowed therapeutic agents to be released freely into a wound, but constrained exudate to an absorbent patch integrated with a permeable epithelial layer of a diffusion dressing. A significant improvement in time to heal results, with no impediment to the quality of care created.

Accordingly, in one embodiment of the present invention, a therapeutic dressing is provided including a permeable bottom layer, a non-permeable upper layer sealed about the periphery to the permeable layer to form at least one reservoir, an absorbent patch integrated with the permeable layer, a hermetic seal attached to the underside of the permeable layer; and a filling interface in communication with the reservoir. The dressing is filled with a therapeutic agent and wherein such agent is subsequently diffused through the permeable layer onto the skin of a patient. In one embodiment, the absorbent patch absorbs exudate from a wound. In this embodiment, the absorbent patch is adhered to or otherwise attached to the permeable layer and replaces a portion of the permeable layer cut out to incorporate the patch.

In one embodiment, the filling interface includes a fill tab in communication with the reservoir the fill tab including a self-sealing orifice adapted to accept a therapeutic agent. In this embodiment, the filling interface communicates with the reservoir through one or more conduits opening into the reservoir. In one embodiment, the absorbent patch includes blown fibers. In one embodiment, the absorbent patch includes a backing layer that prevents tissue growth into its porous surface. In this embodiment, the backing layer is attached to the permeable layer. In one embodiment, there are multiple reservoirs, each reservoir including a self-sealing orifice or valve.

In one embodiment, the agent delivery system further includes an encapsulated canister containing therein a volume of therapeutic agent under pressure, the canister integrated with a plunger mechanism including a sharp tip for puncturing the canister to release the therapeutic agent into the reservoir.

According to an aspect of the present invention, a method is provided for filling a diffusion reservoir in a therapeutic dressing with a therapeutic agent and includes the steps, (a) coupling a vessel containing a therapeutic agent to a self-sealing orifice on the therapeutic dressing, (b) releasing the therapeutic agent into the diffusion reservoir under a low pressure, and (c) uncoupling the canister from the self-sealing orifice sealing the orifice. In one aspect of the method, the vessel is pressurized before application.

In one aspect, in step (a), the vessel is a pressurized canister including a threaded cap with a seal-puncture aperture for breaking the seal on the canister and a hollow needle for delivering the therapeutic agent into the self-sealing orifice on the therapeutic dressing. In a variation of this aspect, in step (b), the threaded cap with the seal-puncture aperture is rotated to break the seal on the canister. In one aspect, the therapeutic agent is a gas. In another aspect, in step (a), the vessel is a pressurized canister with a release valve and a clear delivery tube culminating to a hollow needle for delivering the therapeutic agent into the self-sealing orifice on the therapeutic dressing.

### BRIEF DESCRIPTION OF THE DRAWING FIGURES

FIG. 1 is a perspective view of a diffusion dressing according to an embodiment of the present invention.

FIG. 2 is a partial cut view of diffusion dressing of FIG. 1.

FIG. 3 is a perspective view of a pre-pressurized canister used to deliver therapeutic agents into an internal reservoir of diffusion dressing according to an embodiment of the present invention.

FIG. 4 is a perspective view of a pre-pressurized vessel used to deliver therapeutic agents into an internal reservoir of diffusion dressing according to another embodiment of the present invention.

FIG. 5 is a perspective view of a multi-reservoir diffusion dressing according to another embodiment of the present invention.

### DETAILED DESCRIPTION

The inventors provide a diffusion dressing and methods for maintaining controlled release of therapeutic agents there from to treat wounds. The present invention will be described in enabling detail using the following examples, which may describe more than one relevant embodiment falling within the scope of the present invention.

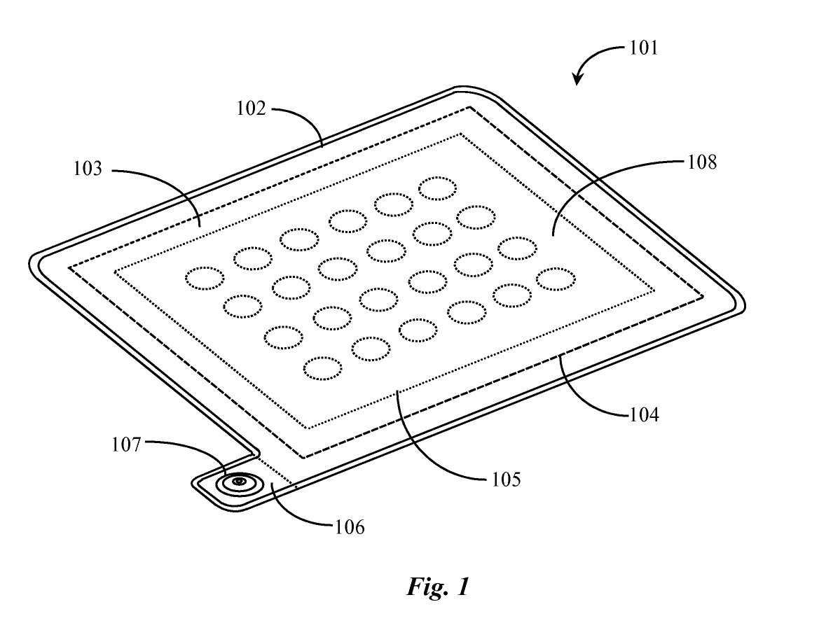

FIG. 1 is a perspective view of a diffusion dressing 101 according to an embodiment of the present invention. Dressing 101 is adapted as a diffusion dressing capable of dispensing therapeutic agents such as a gas, for example, into a tissue wound. Dressing 101 may be applied to a wound for the purpose of delivering a therapeutic agent, for instance a therapeutic gas, to the tissue to be treated. The dispensing of therapeutic agent from the dressing occurs through a diffusion process that allows the tissue to be exposed to the therapeutic agent for an extended period of time, typically days.

Dressing 101 includes may include several layers of material that are sandwiched together to form at least one reservoir for holding the therapeutic agent or agents. Dressing 101 includes, in this embodiment, a bottom layer or “diffusion layer” 102. Diffusion layer 102 is the layer of dressing 101 that makes intimate contact with the skin or damaged tissue of a patient. Diffusion layer 102 may be manufactured of a sterile material that is permeable to a degree that allows diffusion of the therapeutic agent from the reservoir of the dressing into the tissue under low pressure. The material used to form layer 102 may be a continuous sheet of perforated or non-perforated material.

Dressing 101 includes an absorbent patch 105 that may be integrated into diffusion layer 102. Absorbent patch 105 is adapted to absorb biological exudate that may ooze from a healing wound. In one embodiment, a cutout window the same or similar size as the absorbent patch is created in the diffusion layer. In this aspect, absorbent patch 105 may be aligned to the window and adhered into place using a suitable adhesive. Absorbent patch 105 may comprise a thin sponge-like material that is manufactured from non-woven or blown fibrous material. The patch may be cut or formed to any desired shape that conforms appropriately to the overall shape of a common wound dressing. In this case the shape is rectangular. In one embodiment, patch 105 includes a boundary layer or backing layer (not illustrated) that is adapted to protect against tissue growth into its porous surface and may be adhered to the diffusion layer 102.

In one embodiment, diffusion dressing 101 includes an adhesive band seal 104. Band seal 104 may be disposed about the perimeter of dressing 101 on the underside of diffusion layer 102. The dimensions of band seal 104 may vary in terms of width and thickness. The band seal is adapted to provide a substantially hermetic seal that protects the wound from external ambient air, pathogens, and bacteria that may infiltrate the wound area.

Dressing 101 includes a non-permeable cover layer 103. Cover layer 103 is attached, in a preferred embodiment to the diffusion layer the attachment method including but not limited to sealing the two layers together about a shared perimeter to form a reservoir using heat or silicone sealing processes. The area of space formed between layers 103 and 102 form a therapeutic reservoir 108 adapted to contain and diffuse any one or combination of different therapeutic agents infused into the reservoir. Therapeutic agents may include but are no limited to a gas like oxygen or carbon dioxide, a gel, or an emulsion containing a gas. The emulsion may or may not include additional therapeutic drugs with the contained gas. The diffusion rate of dressing 101 depends in part upon materials composition, porosity, and geometry of diffusion layer 102.

Therefore the active diffusion rates for dressings of the type described may be adjustable through regulation of the fore mentioned attributes or features.

In one embodiment of the present invention, dressing 101 includes a special filling tab 106. Filling tab 106 is integrated into the non-permeable layer 103 and the bottom or diffusion layer 102 and is in intimate communication with reservoir 108 through one or more conduits formed by the attachment to dressing 101. Filling tab 106 may be integrated into the larger reservoir (108) of dressing 101 by having one of its edges sharing any unsealed periphery edge of the dressing and having three periphery edges sealed in like manner to that of the larger reservoir body. The filling tab 106 includes, in this embodiment, a self-sealing orifice or port 107. Orifice 107 is a self-closing valve that is described in more detail layer in this specification. Fill tab 106 may act as conduit for administration of one or more therapeutic agents into dressing reservoir 108 via one or more conduits that connect the interior chamber of the filling tab to the major reservoir of the diffusion dressing. In one embodiment, filling tab 106 includes a unique chamber structure that can be manipulated to dispense therapeutic agents from the filling tab into the reservoir of the diffusion dressing.

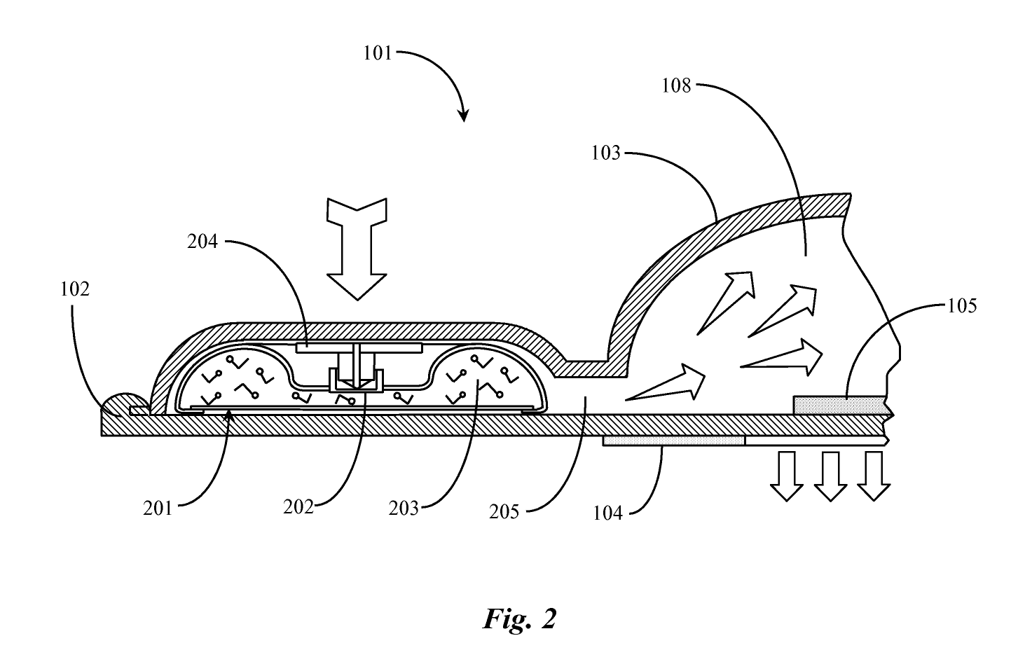

FIG. 2 is a partial cut view of diffusion dressing 101 of FIG. 1. In this example, diffusion dressing 101 includes a mechanical plunger interface in filling tab 106 that is adapted to enable “on board” charging of the diffusion dressing according to one embodiment of the present invention. In this example, diffusion dressing 101 includes filling tab 106 described further above. The filling tab is represented by element number 201 because of inclusion of a pre-fabricated chamber 203 and plunger architecture integrated into the chamber architecture. Chamber 203 is pre-filled with one or more therapeutic agents and then sealed for later use. Chamber 203 may comprise a single piece or multi-piece construction of pliable but non-permeable material such as a polymer or film.

In this example, chamber 203 is of a size and shape that may contain a sufficient volume of therapeutic agents at a higher pressure to fill the entire volume of reservoir 108 at lower pressure. In this example, chamber 203 is in the form of an annular “puck” shaped canister. The material used to form canister 203 is sufficiently strong to hold the higher pressures relative to a gaseous or foam-based therapeutic agent. In one embodiment chamber 203 is constructed of a cold-formed, thin-walled aluminum. In this embodiment, a plunger interface 204 is provided that is integrated into the wall of chamber 203 at a strategic area 202 where the chamber will be punctured to release agent.

A plunger shaft and point is integrated into plunger interface 204 and is orientated toward the thinner wall of strategic area 202.

Prior to activation, the volume in canister 203 is pre-filled with therapeutic agent through the plunger mechanism or via another method to which the introduced agent can be contained indefinitely within chamber 203 at a relatively higher pressure than would be required for diffusing the agent into a wound. The point of filling may incorporate a self-sealing valve or an opening that once punctured may be resealed by self-closing properties of the valve or by strategic abutment of a sealing surface built onto the plunger shaft. In this example, plunger interface 204 may be depressed to activate release of the therapeutic agents into the larger reservoir. The thin wall of the chamber is pierced at strategic area 202 releasing the pressurized contents of chamber 203. The contents of the chamber may translate through one or more small openings (not illustrated) placed strategically into the plunger shaft and then may migrate through one or more conduits into reservoir 208. In alternative embodiments no such passages are required. A single conduit from the filling tab into the larger reservoir is all that is required to introduce the agents into the larger reservoir of diffusion dressing 101.

In one embodiment, therapeutic agents are released under mild pressure into the major reservoir 108 though a conduit 205 integral to the non-permeable upper layer 103 of the dressing. The therapeutic agents are then diffused through the exudate-absorbing material patch 105 and permeable diffusion layer 102 into the wound according to the direction of the arrows. The fill rate may be tailored to suit the size and shape of the dressing by varying the diameter of the orifice in the plunger shaft, or in other embodiments, the number of orifices in the shaft. The materials used to make plunger interface 205 may be one or a combination of plastics, aluminum, or other durable materials capable of puncturing thin walled aluminum.

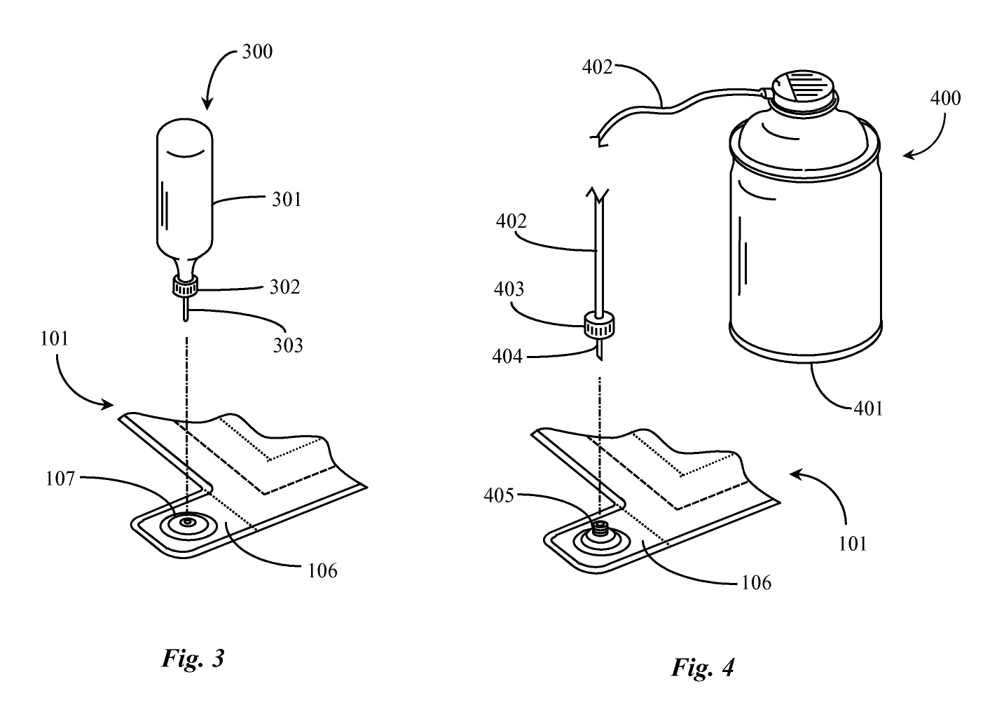

FIG. 3 is a perspective view of a pre-pressurized canister 300 used to deliver therapeutic agents into an internal reservoir of diffusion dressing 101 according to an embodiment of the present invention. In this example, self-sealing valve 107 is formed of a soft rubber disk shaped membrane with a thin washer-shaped retainer ring that may be thermally formed and made integral to the non-permeable upper layer 103 of diffusion dressing 101. In one embodiment, valve 107 may be made integral to the underside of the non-permeable material of the dressing.

Self-sealing valve 107 may be engaged by apparatus associated with canister 300, namely a seal cap 302 formed with a hollow delivery tube 303. As canister 300 is removed, the rubber disk of valve 107 will again contract around the pierced opening reforming the seal to the major reservoir 108 preventing back flow of therapeutic agent out of the reservoir. Canister 300 includes, in this example, a canister body 301, seal cap 302, and hollow fill tube 303. Canister body 301 is of a size and shape sufficient to hold an amount of therapeutic agent under higher pressure that is more than sufficient for filling the entire reservoir volume of a diffusion dressing. Body 301 and the associated hardware may be made from stainless steel, aluminum, or a durable polymer.

Canister body 301 may include a roll-formed thread in the necked-down portion thereof to which like threaded seal cap 302 may be threadably connected. Body 301 may include a small seal of relatively thin wall that is adapted to be punctured by an aperture on the seal cap (aperture not illustrated). The pitch of the threaded cap might be such that a single revolution of the cap in a clockwise (right hand thread) or counter clockwise (left hand thread) is sufficient to puncture the thin wall seal on canister body 301. Another revolution of cap 302 in the same direction might then reseal the canister body preventing further release of agent via abutment against an interior seal (not illustrated) on the underside of cap 302, which will shut off the release of therapeutic agent.

In this way, apparatus 300 can be used repeatedly until all of the contents are expended, which total volume may be sufficient to fully charge multiple diffusion dressings. In practice, a user may insert device 300 into fill interface or valve 107 of dressing 101, and then twist seal cap 302 in the correct direction, piercing the seal on canister body 301. The released agent traverses down fill tube 303 and into the reservoir of the dressing. The canister body can then be re-sealed by further twisting of the cap in the same direction until the “cap seal” engages the punctured opening with sufficient pressure afforded by the new position of the threaded cap.

FIG. 4 is a perspective view of a pre-pressurized vessel 400 used to deliver therapeutic agents into an internal reservoir of diffusion dressing 101 according to another embodiment of the present invention. In this example, a vessel 400 is provided and includes a vessel body 401 that is significantly larger in volume than canister body 301 described above. Vessel body 401 may include a pressure release cap 406 and delivery tubing 402. Cap 406 includes a manually operated button or tab mechanism that releases the therapeutic agent into a variably continuous length of pliable clear tubing 402. Tubing 402 does not have to be clear to practice the present invention. The tube may be opaque or may be of any color or opacity without departing from the spirit and scope of the present invention.

In one embodiment, tubing 402 has a threaded end cap 403 that may be altered in function somewhat from the seal cap described further above. End cap 403 may include a hollow delivery needle or tube 404. The end cap may then be threaded to a fill device 405 that is incorporated, in like manner, onto the fill device as noted previously. Cap 403 includes an interior lip seal (not illustrated) for sealing off the flow of therapeutic agents from vessel 406.

In this example, a modified fill interface or valve 405 is provided. Cap 405 includes an externally threaded pop-up stem that is threaded to accept internally threaded cap 403 with fill needle 404 protruding there from. The seal on the underside or interior of cap 404 seals against leakage of therapeutic agents from the diffusion dressing after fully charging the dressing. This seal is affected by threading cap 403 onto stem 405 and then tightening the cap to prevent backflow of gases. Upon insertion of the hollow needle into the threaded neck of the fill device 405, a seal may be punctured releasing the therapeutic agent into the device. Unlike the end cap of the device of FIG. 3, this end cap may be attached once and may remain so, for the duration of the wound treatment.

In one embodiment, vessel 400 might also include a shut-off valve (not shown) integrated in-line, with tubing 402. A shut-off valve might be used to intentionally interrupt the delivery of therapeutic agent to a dressing. By twisting the shut-off valve to an off position, the user may change-out an expended vessel 401 for a new vessel. It may also afford a user to change-out a wound dressing that is need of repair or replacement, according to one embodiment of the present invention. Vessel body 401 is adapted be used on a wound dressing while the therapeutic agents are diffusing through the depressing onto a wound until all of or a prescribed amount of agent has been used. In one embodiment a patient or responsible aid may manually dispense the therapeutic agents from vessel body 401 into the dressing by manually depressing cap 406 as many times as is required to sufficiently saturate the wound via the diffusion process.

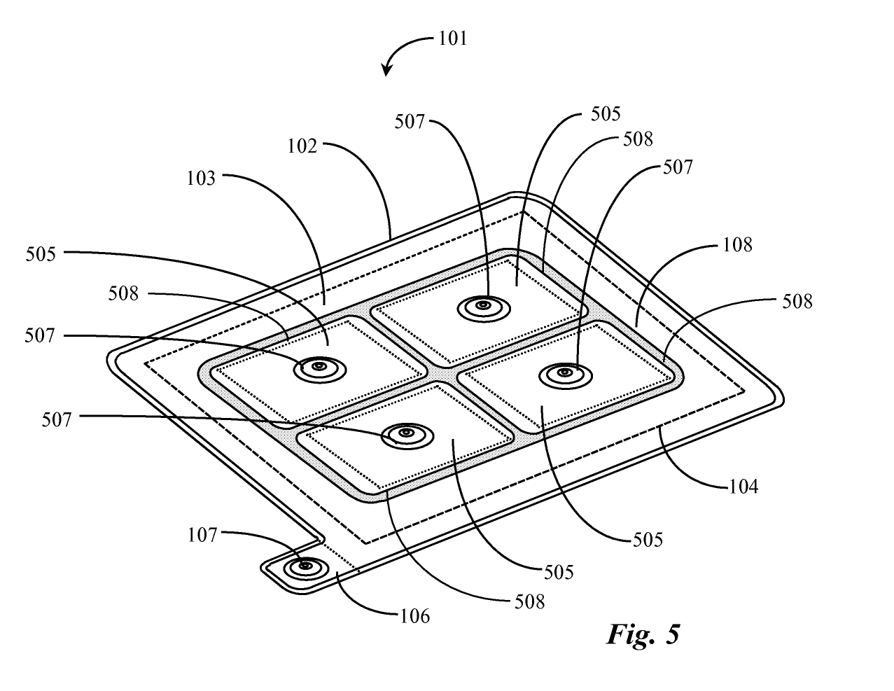

FIG. 5 is a perspective view of a multi-reservoir diffusion dressing 501 according to another embodiment of the present invention. In this example, diffusion dressing 501 includes multiple isolated reservoirs 508 instead of a single reservoir. Reservoirs 508 may be formed by thermally fusing non-permeable upper layer 103 to diffusion lower layer 102 in such a manner as to define the boundaries of the reservoirs. Having multiple reservoirs on a single dressing allows a variety of medicated or non-medicated therapeutic agents to be diffused into a wound.

In this example, each reservoir 508 may include an exudate absorbent patch 505. The dressing may be customized to a variety of wound types and therapeutic substance areas. Each individual reservoir 508 includes a reservoir filling or charging interface or valve 507. Filling interface 107 and fill tab 106 may not be required in this embodiment as each reservoir 508 includes a fill valve.

It will be apparent to a skilled artisan that the embodiments described above are exemplary of inventions that may have greater scope than any of the singular descriptions. There may be many alterations made in these examples without departing from the spirit and scope of the invention. For example, the size and shape may vary widely, depending upon the area of the body the dressing may be applied. The material compositions of the filling devices and or the wound dressings layer construction and composition, as illustrated in this disclosure, may vary depending on the application area and the environment and therapeutic agents to which they may be exposed. These and many other features may change in different embodiments.

It will be apparent to one with skill in the art that the diffusion system of the invention may be provided using some or all of the mentioned features and components without departing from the spirit and scope of the present invention. It will also be apparent to the skilled artisan that the embodiments described above are specific examples of a single broader invention that may have greater scope than any of the singular descriptions taught. There may be many alterations made in the descriptions without departing from the spirit and scope of the present invention.

What is claimed is:

1. A therapeutic dressing comprising:
a permeable bottom layer;
a non-permeable upper layer sealed about the periphery to the permeable layer to form at least one reservoir;
an absorbent patch integrated with the permeable layer;
a hermetic seal attached to the underside of the permeable layer; and
a filling interface in communication with the reservoir;
wherein the dressing is filled with a therapeutic agent and wherein the therapeutic agent is subsequently diffused through the permeable layer onto a patient.

2. The therapeutic dressing of claim 1, wherein the absorbent patch absorbs exudate from a wound.

3. The therapeutic dressing of claim 1, wherein the absorbent patch is adhered to or otherwise attached to the permeable layer and replaces a portion of the permeable layer cut out to incorporate the patch.

4. The therapeutic dressing of claim 1, wherein the filling interface includes a fill tab in communication with the reservoir the fill tab including a self-sealing orifice adapted to accept a therapeutic agent.

5. The therapeutic dressing of claim 1, wherein the filling interface communicates with the reservoir through one or more conduits opening into the reservoir.

6. The therapeutic dressing of claim 1, wherein the absorbent patch includes blown fibers.

7. The therapeutic dressing of claim 1, wherein the absorbent patch includes a backing layer that prevents tissue growth into its porous surface.

8. The therapeutic dressing of claim 7, wherein the backing layer is attached to the permeable layer.

9. The therapeutic dressing of claim 1, wherein there are multiple reservoirs, each reservoir including a self-sealing orifice.

10. The therapeutic dressing of claim 1, further including an encapsulated canister containing therein a volume of therapeutic agent under pressure, the canister integrated with a plunger mechanism including a sharp tip for puncturing the canister to release the therapeutic agent into the reservoir.

11. A method for filling a diffusion reservoir in a therapeutic dressing with a therapeutic agent comprising the steps:
(a) coupling a vessel containing a therapeutic agent to a self-sealing orifice on the therapeutic dressing;
(b) releasing the therapeutic agent into the diffusion reservoir under a low pressure, and
(c) uncoupling the canister from the self-sealing orifice, sealing the orifice.

12. The method of claim 11, wherein the vessel is pressurized before application.

13. The method of claim 11, wherein in step (a), the vessel is a pressurized canister including a threaded cap with a seal-puncture aperture for breaking the seal on the canister and a hollow needle for delivering the therapeutic agent into the self-sealing orifice on the therapeutic dressing.

14. The method of claim 11, wherein in step (b), the threaded cap with the seal-puncture aperture is rotated to break the seal on the canister.

15. The method of claim 11, wherein the therapeutic agent is a gas.

16. The method of claim 11, wherein in step (a), the vessel is a pressurized canister with a release valve and a clear delivery tube culminating to a hollow needle for delivering the therapeutic agent into the self-sealing orifice on the therapeutic dressing.
